(19) United States
(12) Patent Application Publication
(10) Pub. No.: US 2007/0219488 A1
(43) Pub. Date: Sep. 20, 2007

(54) Active drainage system for use in defined natural or surgically created body cavities or lumina

(76) Inventor: Darius Francescatti, Barrington, IL (US)

Correspondence Address: THOMAS M. FREIBURGER, P.O. BOX 1026, TIBURON, CA, 94920, US

(21) Appl. No.: 11/639,495

(22) Filed: Dec. 15, 2006

Related U.S. Application Data
(60) Provisional application No. 60/751,159, filed on Dec. 16, 2005.

(51) Int. Cl. A61M 29/00

(52) U.S. Cl. 604/96.01

(57) ABSTRACT

Cavity conforming balloons with active pressure gradients are provided to replace passive reliance on gravity flow of fluid from organs, body lumina, cavities and the like for more accurate and timely assessment and analysis of exudates. Diffuse drug delivery to cavities or lumina can be administered as an adjunct to the drainage process, as can application of brachytherapy.

This application claims benefit of provisional application No. 60/751,159, filed Dec. 16, 2005.

### BACKGROUND OF THE INVENTION

The invention concerns apparatus for drainage of liquids from a body cavity or lumen of a living patient, including quantification of liquid flow.

Today, assessment of fluid exudate outflow from the body is reliant upon collection by devices dependent on gravity flow.

This flow is therefore often affected either by position of the patient in relation to the collection device, or by pressure exerted by the body on the subject organ or lumina. Quantitative analysis is dependent on constant repetitive observation by nursing staff, and qualitative assessment follows sampling and subsequent analysis, often long after collection.

A well-known example is urine collection by means of a Foley catheter. Urine pools in the bladder, flows out the Foley through a collection tube into a bag, usually hung on the bed. Nursing staff periodically observes and records volume of urine collected, but dependent on patient position and whether the tube is kinked restricting flow, there is likely a time lag between kidney output to the bladder and collection in the bag. Volume per unit time is therefore inexact. This effect is exacerbated by the fact that the Foley does not completely occupy or reach all the space in the bladder, potentially allowing a pool of urine to accumulate until the level reaches the Foley outlet. This level is very dependent on patient position.

There is therefore need for a system to more immediately accumulate and account for fluid extraction, making volume collection data and qualitative analysis immediately available. Preferably, this data is sensed automatically at the bedside with the results communicated immediately to a remote site, for example a nursing station, for timely use in therapeutic decision-making. This invention addresses this need, and in addition to providing accurate and timely urine analysis and output data, will free nursing staff from most bedside data collection, so they can provide increased patient care.

Although this background is presented using urine collection, this invention has applicability to other drainage procedures as well, for example, wound drainage post operatively. As an adjunct to drainage, therapeutic or opaque imaging agents may be administered to the interior surfaces of a cavity or lumen being drained.

### SUMMARY OF THE INVENTION

One embodiment of this invention comprises a low-pressure, cavity-filling balloon on a flexible catheter shaft, or alternatively, on a rigid wand, depending on the cavity being drained and its accessability. At the distal end of the catheter or wand is the balloon; and at the proximal end is a multi-entry hub for separate connections to lumina within the shaft of the catheter or wand.

The catheter or wand comprises two lumina, one for balloon inflation, and one for fluid withdrawal. Preferably, these lumina are coaxial, although they can simply be parallel. More lumina and other configurations of lumina may be provided for other purposes as described below.

On the outside of the balloon is a continuous surface covering which is capable of expanding with the balloon, and which is permeable or semi-permeable such that the fluids to be extracted, or the agents to introduced, can flow through the layer in response to an actively applied pressure gradient. In an alternate embodiment, the covering is intermittent, and shaped to create channels to decrease resistance to fluid flow of liquids over the surface of the balloon while it is in contact with the cavity or lumen being drained. With channels, the fluids being collected need only pass through the surface covering a short distance, as explained below.

At the proximal end of the balloon where it meets the catheter or wand, the outer layer, and any channels therein, communicate with a lumen in the shaft of the catheter or wand for withdrawal of fluid from the body of the patient. This lumen is in fluid communication with a collection system comprising a collection receptacle external to the body. The other catheter or wand lumen is for pressurizing the balloon. Sensors are situated along the fluid path between the balloon and the collection receptacle, preferably outside the body, such that volume per unit time can be immediately assessed, and if desired, fluid properties can be analyzed. Results can then be transmitted to a remote site, for example a receiver console or nursing station, for immediate evaluation and use in making therapeutic decisions. A vacuum drawn on the extraction lumen provides a flow gradient drawing exuded fluid through the outer balloon surface layer, and channels if any, to the catheter or wand, through the in-line sensors, and into the receptacle.

In use, after the catheter is inserted into the patient and properly situated in the target cavity or lumen, controlled pressure is applied to the pressure lumen of the catheter to inflate the balloon such that it fills the target cavity or lumen. Vacuum is then pulled on the extraction circuit to begin active fluid flow and analysis.

Because empty space within the target cavity outside of the balloon is eliminated, stagnant pooling of exudate in the target cavity or lumen is also eliminated. The pressure gradient created by the vacuum assures fluid extraction is accomplished under all conditions of body attitude or stress in a timely manner. Should the vacuum tend to collapse the thickness of the outer balloon layer, a further lumen can be provided in the catheter or wand which communicates between the proximal hub and the distal-most portion of the outer covering of the balloon in order to provide a vent, relieving the force tending to compress the outer covering. The vent can be used when desired, usually intermittently but possibly continuously, to admit a liquid into the permeable layer to keep the compressible permeable layer expanded or to re-expand it. The liquid could include a therapeutic agent, such as a solution of hydrogen peroxide, in the case of a surgical excision cavity. The more rigid and less compressible the permeable layer, the more a venting liquid flow is useful to maintain a flow of the seroma or other liquid being withdrawn.

As a separate but related embodiment of this invention, and where appropriate, the outer permeable layer can be coated or imbibed with drugs to be diffusely administered to the interior surface tissues of the target cavity or lumen, before insertion into the body. In a still further embodiment, a lumen may be provided in the catheter or wand which communicates between a separate entry at the proximal hub, and sealingly passes through the distal end of the balloon to communicate with the distal-most portion of the outer permeable/semi-permeable surface layer on the balloon. Using this lumen, drugs or other therapeutic agents may be introduced under pressure to bathe the interior tissues of the subject cavity in a flushing manner.

A further embodiment of this invention is drainage in combination with brachytherapy utilizing small sources of ionizing radiation. This embodiment could also include application of therapeutic agents as discussed above, including agents to enhance, vary or retard the therapeutic effect of a prescribed dose of radiation therapy. Lumina or channels can be provided to selectively administer such agents prior to or simultaneously with delivery of radiation treatment, providing localized effect. These and other objects, advantages and features of the invention will be apparent from the following description of preferred embodiments, considered along with the accompanying drawings.

### DESCRIPTION OF THE DRAWINGS

FIG. 1 is a schematic view of the system in relation to a body cavity of the patient.

FIG. 2 is a side view in cross-section of the balloon section of a catheter of the invention.

FIG. 3 is a side view of a balloon showing channels in the outer permeable layer.

FIG. 4A is a transverse section showing channels through the complete thickness of the permeable layer, whereas FIG. 4B is a transverse section through the balloon where the channels are only part way through the permeable layer.

FIG. 5 is a side view in cross-section of an alternate embodiment of the invention.

FIG. 6 is a side view in cross-section showing a further embodiment of the invention including a radiation source.

### DESCRIPTION OF PREFERRED EMBODIMENTS

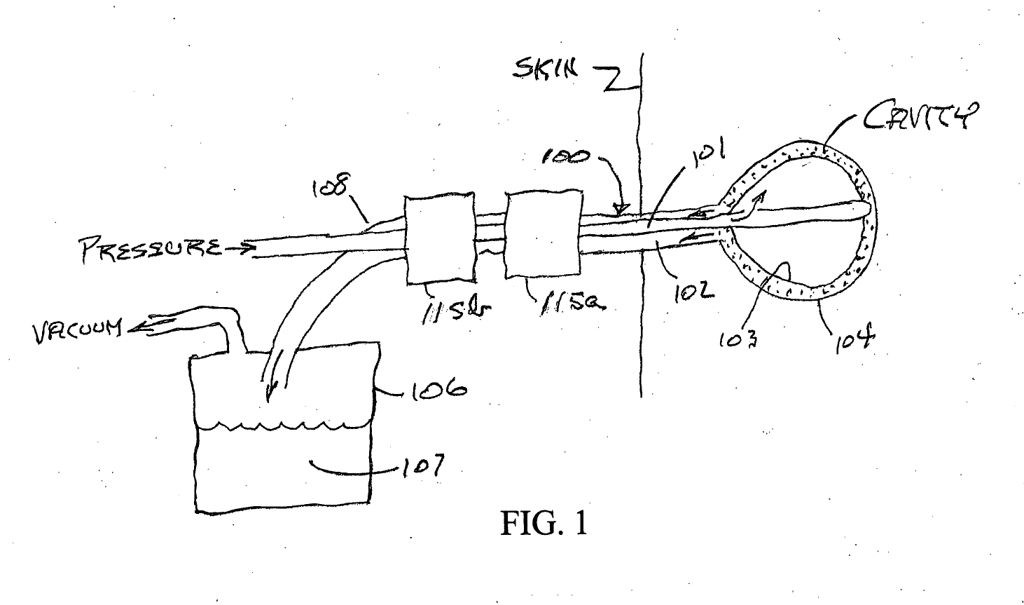

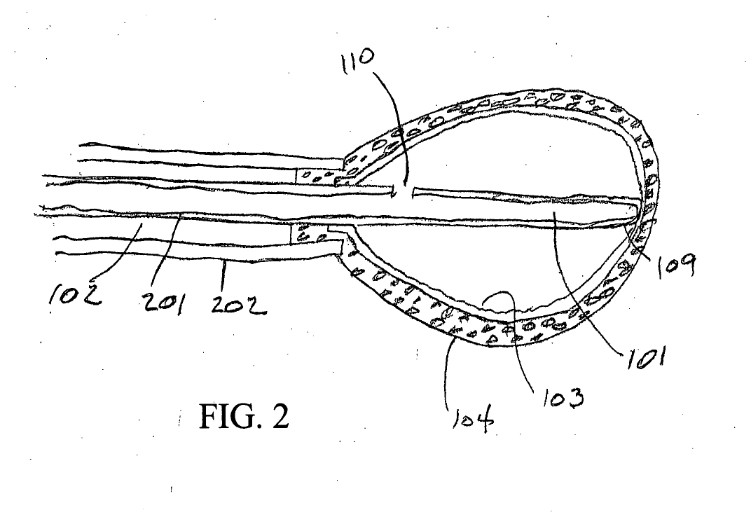

FIGS. 1 and 2 show a wand or catheter 100 comprising a shaft having two coaxial tubes forming channels or lumina, the inner tube 201 forming a lumen 101 for pressure to inflate an impermeable balloon 103, and the outer tube 202 forming a lumen 102 for extraction of exuded fluid (liquid). Outside of, and bonded to the outer surface of the balloon 103 is a permeable or semi-permeable layer 104. The term “permeable” as used in the claims is intended to include both permeable and semi-permeable. The balloon 103 itself is bonded to the shaft portion of the catheter, specifically to the inner tube 201 as shown. The catheter is indicated as inserted into a cavity of the patient and inflated, with the layer 104 in intimate contact with the inner walls of the subject cavity. The inner catheter lumen 101 is connected to a pressure source, for example a squeeze bulb (not shown) outside the body of the patient. The outer lumen 102 passes through sensors 115a for volume, and, in this embodiment, 115b for qualitative analysis as representative of sensors which might be used to assess-volume flow per unit time and quality of exudate. The outer lumen 102 then continues to the liquid collection receptacle 106 shown with liquid 107 therein. The collection receptacle 106 is sealed from the atmosphere to preserve vacuum, which is controlled by pressure regulation methods.

The inner lumen 101 exits the outer lumen 102 at a point 108. If, for example, a turbine type sensor is used to assess fluid flow per unit time, then the sensor 115a might advantageously be placed downstream of the exit point 108, rather than as shown. If quality assessment is spectrographic, the sensor 115b would advantageously pass through a section of the catheter or shaft 100 which is transparent to light. Sensor output can be transmitted, for example wirelessly, to a remote location such as a nursing station where monitoring equipment with display is located.

FIG. 2 shows a cross section in side view of the balloon 103 portion of the catheter or wand. The distal portion 109 of the tube 201 forming the inner lumen 101 extends the length of the balloon 103 to facilitate insertion into the patient. Advantageously, the distal tip 109 is connected to the balloon and rounded to ease insertion of the catheter or wand 100 and to minimize possible damage to the balloon 103 during insertion into the patient. The tube 201 forming the inner lumen 101 has a port 110 opening from the lumen 101 into the balloon 103 to pressurize and expand the balloon. Proximal of the port 110, the proximal end of the balloon is bonded to the tube 201 forming the lumen 101. The permeable layer 104 communicates with the lumen 102 for extraction of exudates. As illustrated in FIG. 2, the outer permeable balloon layer 104 is bonded to the outside of the tube 201 forming the lumen 101, and to the inside of the tube 202 forming the lumen 102.

The concentric tubes 201 and 202 forming the lumina 101 and 102 may be made of polyurethane, for example. The balloon 103 may be made from silicone rubber, for example. The outer permeable layer 104 can be made from open-cell polyurethane foam, for example. Methods to fabricate these sorts of materials into articles like those described are well known in the art.

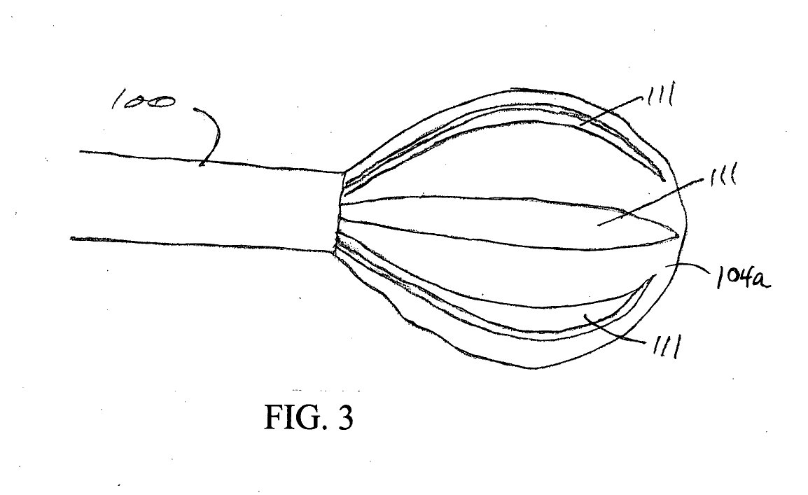

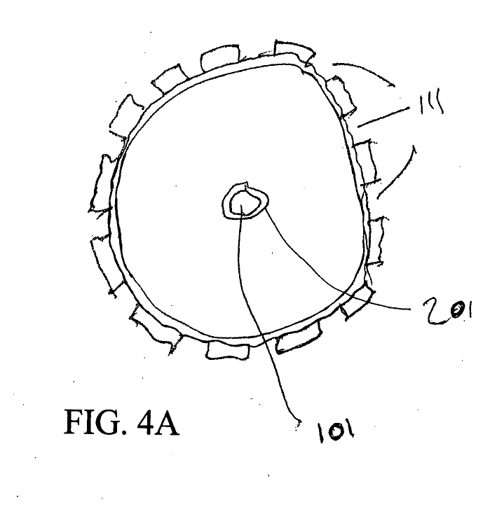

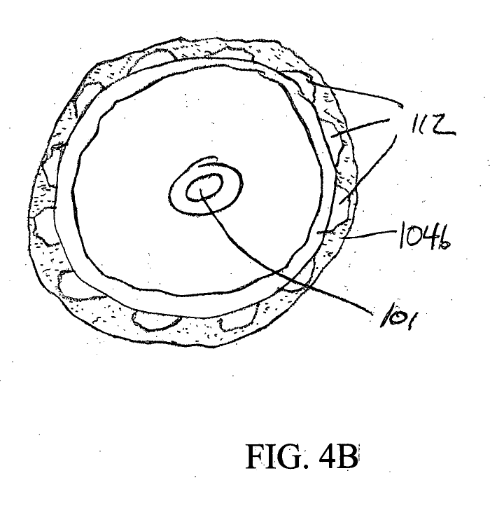

FIGS. 3 and 4A show in side elevation and transverse cross section, the balloon portion of the catheter or wand 100, with another form of permeable outer layer 104a. Channels 111 are formed between and completely through sections of the permeable layer 104a attached to the outside of the balloon 103. These channels 111 may taper down to zero width, merging into a tubular shape where joining tubes 201 and 202 in the way shown in FIG. 2, or optionally, the channels 111 may continue into the bond area such that they communicate directly with the lumen 102. The balloon 103 is visible at the bottom of these channels 111 between sections of the layer 104a. These channels 111 serve to provide increased fluid flow to the lumen 102 at the proximal end of the balloon. With the channels 111, the exudate has only to traverse through the permeable layer a short distance rather than through the permeable layer continuously from the fluid source to the lumen 102. Channel width should be chosen to be narrow enough that tissue of the cavity wall is not attracted sufficiently into the channels so as to block fluid flow.

FIG. 4B shows, again in transverse cross section of the balloon 103 portion of the catheter or wand, channels 112 formed between, but only partially through, sections of the permeable layer 104b attached to the outside of the balloon. These channels 112 again serve to provide increased fluid flow to the lumen 102 at the proximal end of the balloon, and again may optionally continue into the bond area such that the channels 112 communicate directly with the lumen 102. The exudate again has only to traverse through the permeable layer 104b a short distance into a channel 112, rather than through the permeable layer continuously from the fluid source to the lumen 102. This channel construction serves to minimize cavity tissue being drawn into the channels, which could potentially block fluid flow.

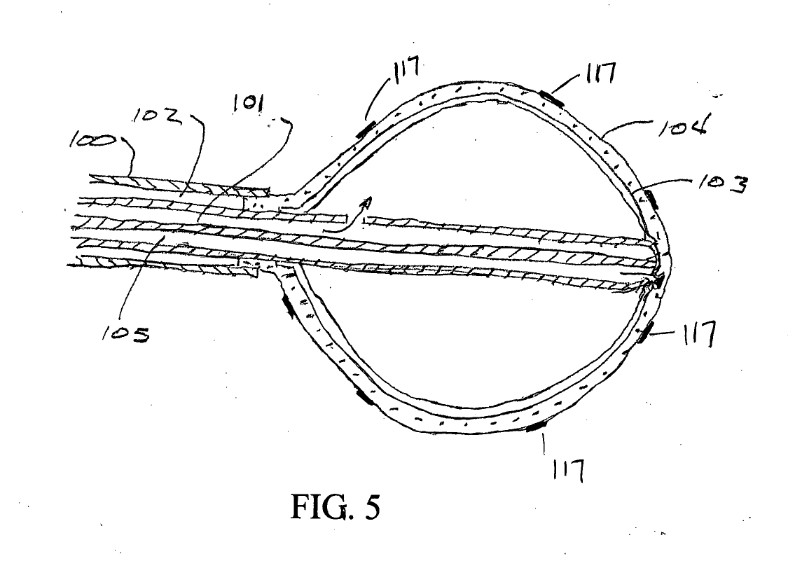

An alternate embodiment of the invention is shown in FIG. 5. FIG. 5 shows the balloon portion of catheter 100 in longitudinal cross-section, including a catheter lumen 105 communicating between the proximal hub and the outer permeable layer 104 after sealingly passing through the distal end of the balloon 103. The lumen 105 is suitable as a vent or for introduction of therapeutic agents under pressure. As discussed above, the vent can be used, intermittently or continuously, to admit a liquid, and the liquid can carry the therapeutic agents.

Another therapeutic adjunct is the application of radiation therapy, particularly in intraoperative situations, for example to the cavity created during a breast lumpectomy where both post-operative radiation therapy and drainage are indicated. Therapy of this sort is described in co-pending application Ser. No. 10/683,885, filed Oct. 13, 2003′, herein incorporated in this specification in its entirety. Suitable miniature electronic x-ray sources are known, for example those of U.S. Pat. No. 6,319,188, “Vascular X-Ray Probe”, adapted as to power capability for the intended application. Where radiation is to be applied, it could be applied via the central pressure lumen 101, or through an auxiliary lumen within the catheter or wand or shaft portion 100 to an axial position within the region encompassed by the balloon 103. In conjunction with radiation therapy and if desired, balloon or catheter mounted dosimeters 117 can be affixed to the exterior of the balloon outer layer 104 or the catheter 100 to monitor and verify the dose delivered, or to adjust radiation delivery parameters in real time during application of the therapy.

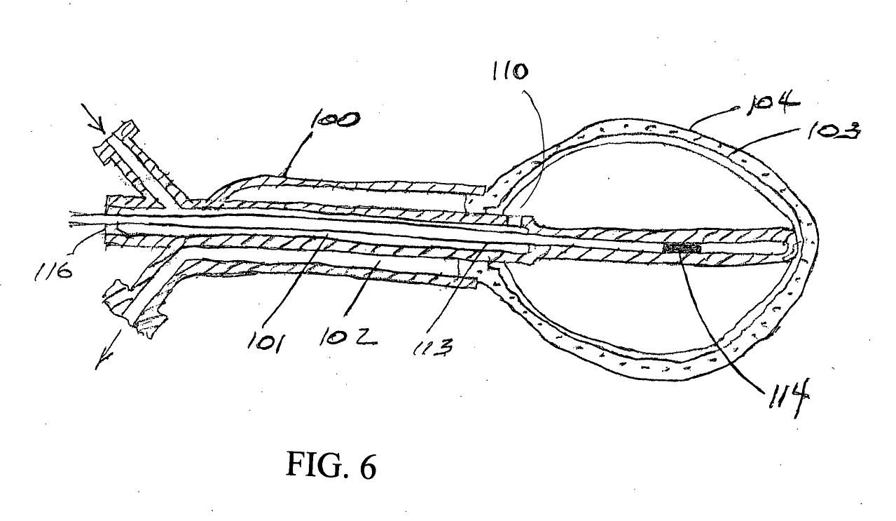

FIG. 6 shows an embodiment comprising introduction of a probe 113 comprising a source of ionizing radiation 114 into the cavity or lumen through the lumen 101 and a seal 116 at the proximate end of the shaft of the catheter 100. Advantageously, the lumen 101 is sized to loosely accommodate the probe 113 at and proximal to the port 110 so as to not interfere with balloon inflation. Distal of the port 110, the lumen 101 may more snugly conform to the probe 113 in order to control the dose distribution of the therapeutic radiation.

In use, the catheter or wand 100 is prepared and inserted into the patient (perhaps with a slippery coating of hydrogel to facilitate insertion), properly situated in the target cavity or lumen, and then controlled pressure is applied, for example by a squeeze bulb (not shown), to the catheter lumen 101 to inflate the balloon 103 such that it fills the target cavity or lumen. Pressure indicators can be used to sense the pressure rise as the balloon fills and occupies the cavity, or alternately, the balloon may be rendered radio-opaque such that cavity filling can be verified by radio-graphic methods. Vacuum is then pulled on the extraction circuit, for example by regulated wall suction applied to the collection receptacle 106, to begin active fluid extraction and analysis. Such analysis might include pH or liquid spectroscopy for example, in addition to volumetric analysis.

If in use the layer 104 loses contact with the issue surrounding the cavity, or the application of vacuum causes partial deflation of the balloon such that it is no longer in contact with the walls of the cavity or lumen, that can be sensed, again by radio-graphic methods if necessary, and the balloon pressure may be increased until contact is again achieved, eliminating dead volume between the cavity and balloon outer surface.

If desired, the outer layer 104 may be coated or imbibed with drugs before the catheter or wand 100 is inserted into the body and the balloon 103 is expanded, those drugs to be suffused into interior cavity or lumen tissue. Such drug delivery can be useful, for example, in treating an abscessed cavity Subsequent drainage can be analyzed over time as the abscess shrinks, and the results used to determine when the drainage system can be safely removed.

With this method and apparatus, effective, active drainage of bodily exudate can be achieved, providing timely data for therapeutic decision making, and facilitating a range of proactive treatment regimens which may be applied, including diffuse drug delivery and brachytherapy. In addition, nursing staff are freed from tedious data logging, and are thus able to handle a greater patient load.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

I claim:

1. A catheter or applicator for draining liquids from within a natural or surgically created body cavity or lumen, with quantitative flow measurement, comprising:
a shaft,
an expandable balloon at a distal end of the shaft,
an outer permeable layer on the outside of the balloon, expandable as the balloon is expanded,
the shaft having liquid withdrawal channel or lumen, the liquid withdrawal lumen being connected to the permeable layer and establishing a flow path for withdrawing liquid,
the shaft further having a lumen for introducing fluid under pressure to inflate the balloon, and
a liquid flow sensor for measuring volumetric flow rate, positioned in the flow path of the withdrawn liquid.

2. Apparatus according to claim 1, wherein the permeable layer comprises a compressible liquid-absorbing layer.

3. Apparatus according to claim 1, wherein the permeable layer comprises a series of channels formed on the outside of the balloon, the channels being capable of receiving liquid, and the liquid withdrawal lumen being connected to receive liquid moving along the channels toward the shaft.

4. Apparatus according to claim 1, wherein the shaft includes a lumen for delivery of therapeutic agents to tissue of the body cavity via the permeable layer.

5. Apparatus according to claim 1, in combination with a source of ionizing radiation, the shaft including a lumen for receiving the source of ionizing radiation into the interior of the balloon, for radiation therapy on tissues surrounding the body cavity.

6. Apparatus according to claim 5, further including a lumen for delivery of therapeutic agents to tissue of the body cavity via the permeable layer.

7. Apparatus according to claim 1, further including a sensor positioned in the path of the withdrawn liquid for qualitative analysis of the withdrawn liquid.

8. Apparatus according to claim 1, wherein the sensor for volumetric flow measurement is secured as a part of the shaft, and including a receiver console for indicating volumetric flow, the receiver console being connected wirelessly to the sensor for volumetric flow.

9. Apparatus according to claim 1, wherein the liquid withdrawn is urine, the catheter distal end being inserted into the bladder of the patient.

10. Apparatus according to claim 1, wherein the liquid withdrawn comprises seroma withdrawn from a surgical wound cavity.

11. Apparatus according to claim 1, including a suction-applying collection receptacle at a downstream end of the liquid withdrawal lumen, drawing liquids from the permeable layer with suction.

12. Apparatus according to claim 11, further including a vent lumen in the shaft and having an end open to a distal part of the permeable layer, to permit resting of the permeable layer when desired.

13. A method for draining liquids from within a natural or surgically created body cavity or lumen, with quantitative flow measurement, comprising:
inserting into the body cavity or lumen a catheter that includes a shaft with an expandable balloon at a distal end of the shaft, with an outer permeable layer on the outside of the balloon, the balloon being deflated on insertion,
with an inflation lumen in the shaft that communicates with the interior of the balloon, inflating the balloon by admitting pressurized fluid to substantially fill the body cavity or lumen with the balloon,
through a liquid withdrawal lumen in the shaft and extending to the permeable layer, applying suction and withdrawing liquid entering the permeable layer from tissue of the body cavity or lumen, and
measuring volumetric flow rate of liquid being withdrawn through the liquid withdrawal lumen.

14. The method of claim 13, further including wirelessly transmitting measured values of volumetric flow rate to a remote location where the values are recorded or displayed.

15. The method of claim 13, further including qualitatively analyzing the liquid as it is withdrawn by the applicator.

16. The method of claim 15, wherein qualitative analysis includes pH or spectrographic analysis.

17. The method of claim 16, further including wirelessly transmitting measured values of volumetric flow rate and qualitative analysis to a remote location where the values are recorded or displayed.

18. The method of claim 13, wherein the outer permeable layer comprises an absorbent, compressible material.

19. The method of claim 18, further including venting the permeable layer while suction is applied, using a venting lumen in the shaft that extends to a distal portion of the permeable layer, venting being accomplished by admitting a liquid.

20. The method of claim 19, wherein the venting liquid includes a therapeutic agent.

21. The method of claim 13, including delivering a liquid therapeutic agent to tissue of the body cavity via the permeable layer.

22. The method of claim 13, wherein the body cavity comprises a patient's bladder, the quantitative measurement of flow rate being a urine flow measurement.

23. The method of claim 22, including qualitatively analyzing the urine, with any contaminants, as it is withdrawn through the liquid withdrawal channel.

24. The method of claim 13, wherein the body cavity is a surgically created cavity from resection of a tumor, the liquid withdrawn being primarily seroma.

25. The method of claim 13, wherein the permeable layer comprises a series of channels formed on the outside of the balloon, the channels being capable of receiving liquid, and the liquid withdrawal lumen being connected to receive liquid moving along the channels toward the shaft.

26. The method of claim 13, further including introducing a source of ionizing radiation through a lumen in the shaft and into the interior of the inflated balloon, and irradiating tissue of the body cavity or lumen surrounding the balloon.
